(19) United States
(12) Patent Application Publication
(10) Pub. No.: US 2024/0122912 A1
(43) Pub. Date: Apr. 18, 2024

(54) A NANOFORMULATION FOR GLIOMA TREATMENT AND PROCESS FOR ITS PREPARATION THEREOF

(71) Applicants: COUNCIL OF SCIENTIFIC AND INDUSTRIAL RESEARCH, New Delhi (IN); JAWAHARLAL NEHRU CENTRE FOR ADVANCED SCIENTIFIC RESEARCH, Bangalore (IN)

(72) Inventors: Madhan Mohan Chandra Sekhar JAGGARAPU, Tarnaka Hyderabad (IN); Eswaramoorthy MUTHUSAMY, Jakkur Bangalore (IN); Tapas Kamar KUNDU, Jakkur Bangalore (IN); Rajkumar BANERJEE, Tarnaka Hyderabad (IN)

(21) Appl. No.: 18/553,657

(22) PCT Filed: Mar. 29, 2022
(86) PCT No.: PCT/IN2022/050317

§ 371 Date: Oct. 2, 2023

(30) Foreign Application Priority Data
Mar. 31, 2021 (IN) 202111015505

(51) Int. Cl. A61K 31/4515; A61K 47/68; A61K 47/69; A61P 35/00

(52) U.S. Cl. CPC A61K 31/4515; A61K 47/6865; A61K 47/6929; A61P 35/00

(57) ABSTRACT

The present invention describes the development of a novel, tumor epithelial cell and tumor-associated macrophage (TAM)-targeting, blood brain barrier (BBB) crossing glucose-based nanospheres (CSP). More specifically, the present invention discloses a nanoformulation and/or a composition having anticancer activity comprising of carbon nanosphere (CSP) and a sigma receptor targeting ligand (H8) in the ratio of 1:0.08 to 1:0.2, a complex prepared thereof, a process for preparation thereof and a kit for delivery of the drug molecule or the formulation or the composition to tumor site.

### FIELD OF THE INVENTION

The present invention relates to a nanoformulation for glioma treatment and the process for its preparation thereof. In particular, the present disclosure relates to nanoformulation (CSP-H8 or CH8) comprising carbon nanospheres (CSP) and a sigma receptor targeting ligand (H8). H8 also possess significant anti-cancer activity. Further, the targeting ability of the CH8 towards sigma receptor-moderately expressing tumor epithelial cells as well as tumor associated macrophages (TAMs) proves that the potent carrier is dually targeting in nature. In orthotopic glioma model in mouse, CH8 treatment, and not free H8 treatment, led to increase in survivability and tumor suppression in mice. Further in the present invention, there is provisions for attaching multiple drugs; hence using an additional drug doxorubicin in the final formulation CHD (CH8+DOX) and found that there is increased anticancer effect. The present disclosure also provides a dual drug delivery strategy and kit which can be more useful for efficient tumor regression in sigma receptor expressing cancers.

### BACKGROUND AND PRIOR ART OF THE INVENTION

Sigma receptors (SRs) are integral membrane proteins found in numerous tissues including brain (Guitart et al., 2004; Rousseaux and Greene, 2016). At the cellular level, they are primarily distributed in the cytoplasmic mitochondrial, endoplasmic reticulum, nuclear membranes etc (van Waarde et al., 2015). The widespread SRs in the central nervous system (CNS) are involved in regulating neuroprotection and many other behaviours including memory and movement (Walker et al., 1993). They are over-expressed during all stages of embryogenesis (Langa et al., 2003), as well as in actively proliferating cells (Van Waarde et al., 2010). Deregulation and over-expression of SRs have been reported to be associated with invasive and metastatic phenotype of aggressive cancers such as breast, colorectal, prostate and brain cancers (Vilner et al., 1995). Owing to the 10-fold higher expression of SR in tumor cells, SR ligands can selectively target tumor cells and induce tumor-cell selective apoptosis (Van Waarde et al., 2010).

At higher concentrations, clinically approved SR ligands like Pentazocine, Haloperidol, Phenothiazines and N-(1-benzylpiperidin-4-yl)-4-iodobenzamide (4-IBP) have shown to inhibit cell proliferation in brain cancer cells through intrinsic pathway of apoptosis (Gil-Ad et al., 2004). A reduced derivative of haloperidol was shown to induce better apoptotic effect in SR-overexpressing cancers compared to Haloperidol (Brent et al., 1996). In order to increase the apoptosis inducing ability of Haloperidol, our group synthesized a cationic lipid conjugated with tertiary —OH of Haloperidol and reported that C8 conjugate of Haloperidol (H8) could act as a potential anticancer drug (Pal et al., 2011). The cellular entry is enhanced owing to the presence of cationic lipid chain, although such modification did not affect the affinity of modified haloperidol (H8) moiety towards SRs.

Glioblastoma multiforme (GBM) is an aggressive malignancy that develops due to the cancerous growth of glial cells in the brain and has very poor prognosis (Phillips et al., 2006). GBM cells tend to grow aggressively forming appendages into the surrounding brain tissue thus making surgical resection difficult and is hence followed by radiation or chemotherapy or both, thus increasing, though rare, cases of extended survivability (Shergalis et al., 2018). Tumor microenvironment (TME) in GBM is composed of lymphocytes, glial stem cells and also tumor associated macrophages (TAMs) (Gronseth et al., 2018; Quatromoni and Eruslanov, 2012). Glial stem cells produce periostin which helps in recruiting TAMs from peripheral blood to GBM tumor environment and it helps in maintaining the M2 subtype of TAMs for GBM tumor progression (Wu et al., 2015). Recent studies show that pleiotrophin secreted from TAMs stimulates glioma stem cells (GSCs) and indirectly involves in promoting tumor growth (Shi et al., 2017). Through these modulations, tumor cells develop local immunosuppressive micro environment that helps from immune surveillance of host immune system (Bloch et al., 2013). All these factors making brain cancers as a challenging disease to treat. Besides this, the blood brain barrier (BBB) which acts as a physical and electrostatic barrier limits the brain permeation to therapeutics making the treatments ineffective and is also responsible for the clinical failures of many effective and potential drugs (Liu et al., 2012). Thus, the development of drugs that can cross the BBB is limited because of the challenges associated with the transport of molecules across it (Liu et al., 2012). The present disclosure accordingly relates to providing tumor mass-targeting delivery system with an aim to overcome or at least to alleviate one or more of the above-mentioned disadvantages of the existing art.

So far, the nanoparticle delivery methods have been shown to be effective method used for the treatment of CNS diseases (Upadhyay, 2014). Carbon nanoparticles are a class of low dimensional materials and owing to their small size, there are chances to probe the nanomaterial, and control biological process which makes them promising candidate materials for use in biomedical applications (Ediriwickrema and Saltzman, 2015). As a new emerging material in carbon nanoparticles, carbon nanospheres received much attention due to their excellent bio-compatibility in various biomedical applications as a drug carrier (Jiang et al., 2017). Due to their BBB crossing ability carbon nanospheres derived from the glucose can be used as a drug carrier to target brain related diseases including cancer.

The present discloser describes the delivery of H8 to in situ glioma by electrostatically conjugating it to the BBB-crossing CSP and effective increase in survivability of glioma-associated orthotopic mouse model through dual-targeting to tumor associated macrophages (TAMs) and tumor endothelial cells.

### OBJECTIVES OF THE INVENTION

The primary objective of the present invention is to develop a nanoformulation for glioma treatment and the process for its preparation thereof. The invention provides a potent therapeutic strategy against aggressive glioblastoma. Herein, a carrier made of glucose-based carbon nanosphere (CSP) is used to cross blood brain barrier (BBB) to reach brain and by using a modified sigma ligand H8 with adequate anti-cancer activity, the nanosphere induced targeted killing of sigma receptor moderately expressing glioma cells.

In addition, the targeting ability of the nanosphere towards sigma receptor expressing tumor associated macrophages and killing them resulted in increased survivability in glioma bearing mice.

### SUMMARY OF THE INVENTION

Accordingly, the present invention provides a nanoformulation for glioma treatment and the process for its preparation thereof. The invention discloses a dual-targeting system that could target tumor cells as well as TAMs in glioblastoma (GBM) using nano-conjugates formed by surface modification of glucose-based carbon nanospheres with an sigma receptor (SR) targeting ligand, H8. The compound, H8 itself acts as ligand as well as anti-proliferative drug to the SR expressing tumor cells. The system specifically targets the SR expressing tumor cells and tumor accompanying cells in the tumor microenvironment. Our in vitro and in vivo results clearly revealed the repurposing of HP-alike neuropsychotic agents towards achieving receptor-targeted dual acting strategy against tumor cells and TAMs in GBM tumor microenvironment. Thus, this strategy provides a worthwhile approach enabling effective tumor regression and enhanced survivability in GBM and other solid tumors.

In an aspect of the present invention, the present invention discloses a nanoformulation having anticancer activity comprising a complex of a carbon nanosphere (CSP) and a sigma receptor targeting ligand (H8) in a ratio of 1:0.08 to 1:0.2.

In another aspect of the present invention, the present invention discloses a process for the preparation of a nanoformulation having anticancer activity comprising a complex of a carbon nanosphere (CSP) and a sigma receptor targeting ligand (H8) in a ratio of 1:0.08 to 1:0.2, comprising the steps of:
i.) Providing
   a. N-(carboxymethyl)-N-methyl-N-octyloctan-1-aminium chloride
   b. N-[(Dimethylamino)-1H-1,2,3-triazolo-[4,5-b] pyridine-1-ylmethylene]-N-methylmethanaminium hexaflurophosphate N-oxide (HATU)
   c. β-Alanine-Haloperidol conjugate
   d. CSP (Carbon nanosphere)
ii.) Dissolving N-(carboxymethyl)-N-methyl-N-octyloctan-1-aminium chloride as obtained in step (i)(a) in dry dimethylformamide (DMF), and stirred over an ice bath to obtain a mixture;
iii.) Adding N-[(Dimethylamino)-1H-1,2,3-triazolo-[4,5-b] pyridine-1-ylmethylene]-N-methylmethanaminium hexaflurophosphate N-oxide (HATU) as obtained in step (i)(b) to the mixture as obtained in step (ii) to obtain a reaction mixture;
iv.) Dissolving β-Alanine-Haloperidol conjugate as obtain in step (i)(c) in dry dimethylformamide (DMF) to obtain a conjugate mix;
v.) Adding diisopropylethylamine (DIPEA) to the conjugate mix obtained in step (iv) followed by dropwise addition of the mix to the reaction mixture obtained in step (iii), until reaction mixture become slightly basic;
vi.) Stirring the reaction mixture as obtained in step (iv) for 40-50 hours;
vii.) Dissolving dichloromethane (DCM) in the reaction mixture as obtained in step (v) followed by washing with 1N-HCl, water and brine, drying with anhydrous Na₂SO₄, evaporating, and purifying to obtain H8;
viii.) Dissolving H8 as obtained in step (vi) in methanol, further the solution is added to CSP as obtained in step (i) (d) and keeping the conjugate under bath sonication for 5-10 minutes followed by stirring for 10-12 hours at room temperature to obtain a nanoconjugate mixture;
ix.) Centrifuging the nanoconjugate as obtained in step (vii) for 10 minutes at 20-30□, to obtain a CSP nanoconjugate pellet.

In an embodiment of the present invention, the nanoformulation is conjugated with an additional drug, wherein the additional drug is selected from a group of anticancer drugs comprising of doxorubicin, gemcitabine, temozolomide, carmustine, and everolimus.

In an embodiment of the present invention, the nanoformulation is useful for targeting tumor epithelial cell (TEC) and tumor associated macrophages (TAM) in glioblastoma mass. In another aspect of the present invention, the present invention discloses a complex of general formula:

CSP-H8-D
wherein, the CSP represents a carbon nanosphere; the H8 represents a sigma receptor targeting ligand and the D represents a potent drug;
wherein, the CSP is conjugated with the H8; and CSP-H8 conjugate is covalently or non-covalently linked to the potent drug D.

In an embodiment of the present invention, the potent drug D is a hydrophilic or hydrophobic anticancer agent selected from the group comprising of doxorubicin, gemcitabine, carmustine, everolimus, and temozolomide.

In an embodiment of the present invention, the carbon nanosphere (CSP) and the sigma receptor targeting ligand (H8) are present in a ratio of 1:0.08 to 1:0.2.

In an embodiment of the present invention, the complex is useful for targeting tumor epithelial cell and tumor associated macrophages in tumor or glioblastoma mass.

In another aspect of the present invention, the present invention discloses a process for preparing a complex of general formula:

CSP-H8-D,
the process comprises the steps of:
(i) conjugating a carbon nanosphere (CSP) with the sigma receptor targeting ligand (H8) to form a CSP-H8 or CH8 nano conjugate;
(ii) conjugating a potent drug D to the CH8 by mixing CSP-H8 or CH8 nano conjugate with an alcoholic solution of the potent drug D and stirring for a period sufficient to ensure linking of D to CH8 with a maximum limit maintaining a CH8:D ratio of 1:0.2.

In an embodiment of the present invention, the period of stirring is 7-15 hours, preferably 8-12 hours.

In an embodiment of the present invention, the alcohol used is C1 to C3 alcohol.

In another aspect of the present invention, the present invention discloses a tumor or glioblastoma mass-targeting composition, comprising:
a) a carbon nanosphere (CSP), carrying cationic sigma ligand that is a conjugate of cationic lipid; and
b) a haloperidol derivative, as sigma receptor targeting ligand (H8).

In an embodiment of the present invention, the carbon nanosphere (CSP) and the sigma receptor targeting ligand (H8) are present in a ratio of 1:0.08 to 1:0.2.

In an embodiment of the present invention, the composition is useful for targeting tumor epithelial cell (TEC) and tumor associated macrophages (TAM) in glioblastoma mass.

In another aspect of the present invention, the present invention discloses a drug delivery kit for specific delivery of drug molecule to tumor site, having a complex, prepared by conjugating a sigma receptor targeting ligand (H8) to a glucose derived carbon nanosphere (CSP).

In an embodiment of the present invention, the complex is further conjugated with an additional drug, wherein the additional drug is selected from a group of anticancer drugs comprising of doxorubicin, gemcitabine, temozolomide, carmustine and everolimus.

In an embodiment of the present invention, the kit is useful for targeting tumor epithelial cell and tumor associated macrophages for treatment of glioblastoma or tumor mass.

In another aspect of the present invention, the present invention discloses a method of treating tumor or glioblastoma mass by targeting both tumor epithelial cells (TEC) and tumor-associated macrophages (TAM) with a nanoformulation or a composition as claimed in claims 1, 12 and 15 respectively.

### BRIEF DESCRIPTION OF THE ACCOMPANYING DRAWINGS

FIG. 1: Schematic representation of chemical synthesis of H8 and Q8.

FIG. 2 Chemical structures of synthetically prepared target molecule, H8 and its control molecule, Q8.

FIG. 3 Flow cytometer studies with DCFDA staining for untreated 5 μM and 10 μM treatments of CH8 for 16 h in cancerous cells GL261 (left) and U87 (right).

FIG. 4 Apoptosis analysis by FACS in cancerous (GL261, U87); normal cells (CHO and HEK293). Cells were either kept untreated (UT) or treated with CH8 (5 μM) or CQ8 (5 μM) for 24 h followed by apoptosis analysis by FACS study.

FIG. 5 Comparison of in-vivo accumulation of CSP-DiR and CH8-DiR in orthotopic GL261 tumor bearing mice. In-vivo imaging of brain region of mice at 8 hours and 24 hours of treatment.

FIG. 6 Epi-fluorescence images of brains isolated from the mice treated with DiR labelled CSP (a) and CH8 (b) after 8 hours and 24 hours of treatment. DiR distribution in mice brain for respective time points with CSP-DiR and CH8-DiR; c) The graph represents the ex-vivo brain uptake comparison of two treatment groups CSP-DiR and CH8-DiR in 8 hours and 24 hours.

FIG. 7 SR-targeted CSP effectively inhibits orthotopic glioma progression in mice: a) Kaplan-Meier survival analysis of orthotopic glioma bearing mice on treatment with CH8, H8 post 4^{th}, 6^{th}, 8^{th}, 10^{th} and 12^{th} days of tumor cell inoculation; b) Tumor-bearing brains isolated from C57BL/6J mice treated with 5% glucose, H8 and CH8 (5 alternate intraperitoneal injections), after 12 days of inoculation of cells orthotopically into brain through stereotactic surgery; c) Tumor regression curve for heterotopic (subcutaneous) GL261 tumor model of C57BL/6J mice treated with 5% glucose, H8 and CH8 on days 11, 13, 15, 17 & 19 after tumor inoculation; d) GL261 subcutaneous tumors isolated from mice followed by respective treatments for the represented groups after 19 days of tumor inoculation e) Tumor volume regression analysis of indicated treatment groups in subcutaneous glioma tumor model; f) Survival analysis in subcutaneous GL261 tumor bearing mice treated with H8 and CH8. The amount of H8 in both orthotopic and heterotopic tumor models was 8 mg/Kg body weight.

FIG. 8 Comparison of surface markers: a) FACS analysis for expression levels of tumor-associated surface markers on TAMs, isolated from a subcutaneous tumor mouse. Representative images of cytometric analysis of TAMs labelled with antibodies against F4/80, CD68, LY6C and MHCII. TAMs and their corresponding IgG isotype are represented accordingly. b) FACS analysis of SR-expression levels in TAMs and tumor cells isolated from subcutaneous tumor.

FIG. 9 CH8 uptake in TAM: a) FACS analysis of CH8 uptake in TAMs and tumor cells i.e., excluding TAMs obtained from the subcutaneous tumor-bearing mice; b) Flow cytometric analysis of CSP and CH8 uptake in TAMs isolated from tumor-bearing mice.

FIG. 10 MTT for CSP conjugates in GL261 cells (48 h). The accommodation of drug (FDA approved) which is a may be hydrophobic or hydrophilic possible due to lipophilic nature of CSP. Table 1—Hydrodynamic size, Zeta potential and PDI of CSP and its conjugate: CSP represents carbon nanospheres; CH8 (CSP-H8) and CQ8 (CSP-Q8).

### DETAILED DESCRIPTION OF THE INVENTION

Procurement details: The hydrothermal synthesis of CSP, was done by using previous protocol (Selvi et al., 2008). Haloperidol (HP), amberlite Cl⁻ ion exchange resin and MTT reagent were purchased from Sigma Aldrich, India and TAM isolation was done by MidiMACS starting kit having MidiMACS separator (130-042-302), LS columns (130-042-401), CD11b MicroBeads (130-097-142), FITC conjugated antibodies CD68 (130-102-534), F4/80 (130-102-327) Ly-6C (130-102-295) and MHCII (130-102-168) (Miltenyi Biotech Asia Pacific Pte Ltd, Singapore). Dulbecco's modified Eagle's medium (DMEM—Genetix Cat No: CC3004) and propidium iodide (PI), fluoroshield™ with DAPI, Hank's balanced salt solution (HBSS) buffer, Dulbecco's Phosphate Buffer Saline (DPBS), penicillin, streptomycin, kanamycin and fetal bovine serum (FBS) were purchased from Sigma-Aldrich Chemicals, USA. Triton X-100 was obtained from Genetix Brand Asia Pvt. Ltd. (India). Tween-20 was procured from Amresco (USA). Sodium hydroxide (NaOH), xylene and isopropanol were bought from Finar (India). Dimethyl sulphoxide (DMSO) was purchased from Rankem (India). Sodium bicarbonate (NaHCO₃) and glycine were obtained from HiMedia (India). Milli-Q-grade water was used for all of the experiments. NIR dye DiR (part No: 125964) were purchased from Perkin Elmer, USA. 2′,7′-Dichlorofluorescin diacetate (DCFDA) was purchased from Hiclone, India. Column chromatography was done with silica gel (60-120 mesh and 100-200 mesh, Acme Synthetic Chemicals, India). All the other chemicals were acquired from local providers and used without further purification. All the intermediate compounds and final compounds were characterized by ESI mass spectrometry and ¹H NMR. The final compound was characterized by ESI-mass spectrometry, HRMS, ¹H NMR, ¹³C NMR and qualitatively by HPLC.

Cell lines: Brain cancer cells GL261, U87 (National Cancer Institute, USA) and non-cancerous cells CHO and HEK293 were purchased from National Centre for Cell Sciences (Pune, India).

Antibodies: Antibody against SR (ab53852) was purchased from Abcam. Primary antibody (Ki-67 Primary (PA5-19462, Thermo Scientific (USA); 1:100) and secondary antibody (goat anti-rabbit IgG-PE, sc-3739, Santa Cruz; (USA) 1:100) were used for immunofluorescence assay.

Kits: Annexin V-FITC-labeled apoptosis detection kit (Cat No #640914) was purchased from BioLegend.

The present disclosure discloses a nanoformulation having anticancer activity comprising a complex of a carbon nanosphere (CSP) and a sigma receptor targeting ligand (H8) in a ratio of 1:0.08 to 1: 0.2.

The present disclosure discloses a process for the preparation of a nanoformulation having anticancer activity comprising a complex of a carbon nanosphere (CSP) and a sigma receptor targeting ligand (H8) in a ratio of 1:0.08 to 1:0.2, comprising the steps of:
i.) Providing
   a) N-(carboxymethyl)-N-methyl-N-octyloctan-1-aminium chloride
   b) N-[(Dimethylamino)-1H-1,2,3-triazolo-[4,5-b] pyridine-1-ylmethylene]-N-methylmethanaminium hexaflurophosphate N-oxide (HATU)
   c) β-Alanine-Haloperidol conjugate
   d) CSP (Carbon nanosphere)
ii.) Dissolving N-(carboxymethyl)-N-methyl-N-octyloctan-1-aminium chloride as obtained in step (i)(a) in dry dimethylformamide (DMF), and stirred over an ice bath to obtain a mixture;
iii.) Adding N-[(Dimethylamino)-1H-1,2,3-triazolo-[4,5-b] pyridine-1-ylmethylene]-N-methylmethanaminium hexaflurophosphate N-oxide (HATU) as obtained in step (i)(b) to the mixture as obtained in step (ii) to obtain a reaction mixture;
iv.) Dissolving β-Alanine-Haloperidol conjugate as obtain in step (i)(c) in dry dimethylformamide (DMF) to obtain a conjugate mix;
v.) Adding diisopropylethylamine (DIPEA) to the conjugate mix obtained in step (iv) followed by dropwise addition of the mix to the reaction mixture obtained in step (iii), until reaction mixture become slightly basic;
vi.) Stirring the reaction mixture as obtained in step (iv) for 40-50 hours;
vii.) Dissolving dichloromethane (DCM) in the reaction mixture as obtained in step (v) followed by washing with 1N-HCl, water and brine, drying with anhydrous Na₂SO₄, evaporating, and purifying to obtain H8;
viii.) Dissolving H8 as obtained in step (vi) in methanol, further the solution is added to CSP as obtained in step (i) (d) and keeping the conjugate under bath sonication for 5-10 minutes followed by stirring for 10-12 hours at room temperature to obtain a nanoconjugate mixture;
ix.) Centrifuging the nanoconjugate as obtained in step (vii) for 10 minutes at 20-30□, to obtain a CSP nanoconjugate pellet.

The present disclosure provides a nanoconjugate comprising of a nanosphere with a sigma receptor targeting ligand. The sigma receptor targeting ligands are cationic sigma ligands.

The present disclosure relates to a nanoconjugate having a general formula:

CSP-H8

wherein CSP represents a carbon nanosphere and H8 represents a sigma receptor targeting ligand with anti-cancer activity. The present disclosure provides a complex of general formula:

CSP-H8-D

wherein, CSP represents a carbon nanosphere; H8 represents a sigma receptor targeting ligand and D represents a potent drug. Wherein CSP is conjugated with H8; and CSP-H8 conjugate is covalently or non-covalently linked to the potent drug D.

The active agent can be an anticancer drug, for example a hydrophilic or hydrophobic anticancer agent selected from but not limiting to doxorubicin, gemcitabine, carmustine, everolimus, or temozolomide.

The present disclosure provides a composition comprising the complex of general formula:

CSP-H8-DOX

wherein, CSP represents a carbon nanosphere; H8 represents a sigma receptor targeting ligand being a cationic sigma comprising a conjugate of a cationic lipid; and DOX represents a potent drug, wherein CSP is conjugated with H8; and CSP-H8 conjugate is covalently or non-covalently linked to the active agent DOX that represents doxorubicin.

The present disclosure provides a process for preparing a conjugate having a general formula CSP-H8, said process comprises the steps of:
(i) Conjugating a carbon nanosphere CSP with the sigma receptor targeting ligand

The present disclosure provides a process for preparing a conjugate comprising a carbon nanosphere with a sigma receptor targeting ligand, as per the following general scheme:

CSP+H8 [symbol] CSP-H8

CSP-H8 conjugates can be prepared by mixing CSPs in the powder form with the H8 alcoholic solution and stirring for a period sufficient to ensure conjugation to the desired extent. In one embodiment, the period of stirring may be for 7-15 hours, preferably 8-12 hours. The alcohol used to prepare the solution of H8 may be C1 to C3 alcohol.

The present disclosure provides a process for preparing a complex of general formula: CSP-H8-D, said process comprises the steps of:
(i) conjugating a carbon nanosphere (CSP) with a sigma receptor targeting ligand (H8);
(ii) conjugating of a potent drug D to the CH8 can be carried out by mixing CSP-H8 nano conjugate with the alcoholic solution of drug and stirring for a period sufficient to ensure linking to the desired extent. In one embodiment, the period of stirring may be for 7-15 hours, preferably 8-12 hours. The alcohol used to prepare the solution of DOX may be C1 to C3 alcohol.

The present disclosure also contemplates a substitution of doxorubicin with any other anticancer active agent. The anticancer active agent that may be suitable to substitute doxorubicin can be a hydrophilic or hydrophobic anticancer drug. The anticancer active agent for example may be gemcitabine, temozolomide, carmustine, everolimus or the like.

The tumor mass-targeting composition of the present disclosure comprising carbon nanosphere, which carry cationic sigma ligand that is a conjugate of cationic lipid and haloperidol, as sigma receptor targeting ligand, which can target both, tumor epithelial cells (TEC) and tumor-associated macrophages (TAM) in glioblastoma mass inside brain. As the composition has added advantage of its ability to carry additional anti-cancer drugs, the composition shows its ability to target and kill both TEC and TAM, thereby exhibiting dual targeting strategy for effective tumor regression and enhanced survivability rate.

The present disclosure provides a dual drug delivery strategy which can be more useful for efficient tumor regression in sigma receptor expressing cancers. Further in advance the conjugation of additional drug may result prominent therapeutic efficacy.

The inventors of the present disclosure after significant experiments involving substantial human and technical intervention have been able to unexpectedly provide the material for the specific delivery of the drug molecule to the tumor site by conjugating a sigma receptor targeting ligand H8 to a glucose derived carbon nanosphere CSP. The present disclosure provides a material so invented and knowhow for highly selective drug delivery material to all types of SR expressing cancers. The area of medical science is likely to benefit most from the present invention in the area of cancer chemotherapy.

The advantage of the present disclosure is about the specificity of the drug delivery to the glioma region through BBB. Additionally, the nano-conjugate exhibits targeting ability towards sigma receptor expressing tumor epithelial cells and tumor associated macrophages. These findings were hitherto unknown and hence a surprising finding by the inventors of the present disclosure.

In another embodiment the present disclosure provides a composition comprising a conjugate of a carbon nanosphere with a sigma receptor targeting ligand linked for targeting tumor cells and tumor-associated macrophages (TAM). In yet another embodiment the present disclosure provides a composition comprising a conjugate with an additional drug to get much more therapeutic efficiency. In a specific embodiment the present disclosure provides a composition comprising a conjugate of a carbon nanosphere with a sigma receptor targeting ligand linked to doxorubicin as an active agent for targeting tumor epithelial cells (TEC) and tumor-associated macrophages (TAM) in glioblastoma mass.

### EXAMPLES

Following examples are given by way of illustration; therefore, should not be construed to limit the scope of the invention.

### Example 1

### Synthesis of Methyl Dioctylglycinate (a)

Methyl glycinate (0.5 g, 5.6 mmol) was dissolved in 20 mL of dry ethyl acetate in a 50 mL round-bottomed flask fitted with a reflux condenser. Potassium carbonate (1.9 g, 13.77 mmol) and 1-bromooctane (4.278 g, 22.4 mmol) were added, and the resulting mixture was refluxed over an oil bath at 70-80° C. for 12 hours. Then it was cooled and washed with water (2×20 mL) and brine (1×20 mL), dried over anhydrous Na₂SO₄, and evaporated. Column chromatographic purification of the residue using 60-120 mesh silica gel and 10% ethyl acetate-hexane (v/v) as eluent yielded compound (1.29 g, 73.3% yield, Rf=0.6 in 10% ethyl acetate-hexane, v/v).

ESI-MS: m/z=314 [M⁺+1] for C₁₉H₃₉O₂N.

### Example 2

### Synthesis of N-(2-methoxy-2-oxoethyl)-N-octyloctan-1-aminium Chloride (b)

Compound (a) (0.3 g, 0.95 mmol) was dissolved in 5 mL of dry DCM in a 25 mL round-bottomed flask, and excess methyl iodide (3 mL) was added. The reaction mixture was stirred for 4 hours at room temperature. Then it was concentrated and the residue upon column chromatographic purification using 60-120 mesh silica gel and 2% methanol-chloroform (v/v) as eluent, yielding compound as a yellowish gummy liquid (0.1 g, 87% yield, Rf=0.4 in 5% methanol-chloroform, v/v). of the residue followed by chloride ion exchange chromatography (using Amberlite IRA-400 Cl resin and methanol as eluent)

ESI-MS: m/z=329 [M⁺+1] for C₂₀H₄₂O₂NCl.

### Example 3

### Synthesis of N-(carboxymethyl)-N-methyl-N-octyloctan-1-aminium Chloride (c)

Compound (b) (0.2 g, 0.55 mmol) was dissolved in 1:1 ratio of THF and H₂O of 10 mL in a 25 mL round-bottomed flask and added 10 equivalents of lithium hydroxide, further allowed to stir for 3 hours at room temperature. Followed by washing with 2N-HCl (2×30 mL) and extracted with ethyl acetate. The residue was purified by flush column chromatography using 60-120 mesh silica gel and 8% methanol-chloroform (v/v) as eluent to obtain compound (c), as a yellow gummy liquid (69.7% yield, Rf=0.5 in 10% methanol-chloroform, v/v).

ESI-MS: m/z=314.5 for C₁₉H₄₀O₂NCl.

### Example 4

### Synthesis of N-Boc-β-alanine-Haloperidol Conjugate (d)

A mixture of N-Boc-β-alanine (1.35 g, 7.17 mmol), haloperidol (2.24 g, 5.9 mmol), and N,N-dimethyl-aminopyridine (DMAP) (0.53 g, 2.92 mmol) was dissolved in 10 mL of dry DCM in a 50 mL round-bottom flask and stirred in ice for half an hour. To the mixture, DCC (1.5 g, 7.29 mmol) dissolved in 5 mL of dry DCM was added, and the sample was stirred in an ice bath for 1 hour. The reaction mixture was further stirred for 24 hours at room temperature. Then the reaction mixture was filtered. The filtrate was washed with water (2×30 mL) and brine (1×30 mL), dried with anhydrous Na₂SO₄, and evaporated. Column chromatographic purification of the residue using 60-120 mesh silica gel and 1% methanol-chloroform (v/v) as eluent yielded compound (d) as a reddish yellow gummy material (2.85 g, 85.8% yield, Rf=0.6 in 5% methanol-chloroform, v/v).

ESI-MS: m/z=548 [M⁺+1] for C₂₉H₃₆O₅N₂ClF.

### Example 5

### Synthesis of β-Alanine-Haloperidol Conjugate (e)

Compound (d) (1.0 g, 1.83 mmol) was dissolved in 18 mL of dry DCM in a 50 mL round-bottomed flask and stirred over an ice bath for 15 minutes. Then 2 mL of trifluoroacetic acid was added dropwise to the solution. The reaction mixture was further stirred over an ice bath for 4 hours, and then the solution was neutralized using saturated NaHCO₃ solution. The mixture was extracted with DCM (2×15 mL), and the organic layer was dried with Na₂SO₄. Evaporation of the organic layer afforded compound (e) as a reddish-yellow gummy material (0.75 g, 92% yield, Rf=0.4 in 10% methanol-chloroform, v/v, active in ninhydrin charring. As compound (e) was obtained as 95% pure (revealed by TLC), it was directly used for the next step.

ESI-MS: m/z=447 [M⁺30 1] for C₂₄H₂₈O₃N₂ClF.

### Example 6

### Synthesis of H8 (f)

Compound (c) (0.250 g, 0.56 mmol) was dissolved in 5 mL of dry DMF in a 25 mL round-bottomed flask and stirred over an ice bath for 15 minutes. To this, N-[(Dimethylamino)-1H-1,2,3-triazolo-[4,5-b]pyridin-1-ylmethylene]-N-methylmethanaminium hexafluorophosphate N-oxide (HATU) (0.231 g, 0.61 mmol) was added, and stirring was continued for another 30 minutes. Then compound (e) (0.195.5 g, 0.56 mmol) dissolved in 2 mL of dry DMF was added followed by dropwise addition of diisopropylethylamine (DIPEA) until the reaction mixture became slightly basic. The resulting mixture was stirred for 48 hours. Then the reaction mixture was dissolved in 20 mL of DCM, washed with 1N-HCl (2×20 mL), water (1×20 mL), and brine (1×20 mL), dried with anhydrous Na₂SO₄, and evaporated. Column chromatographic purification (using 100-200 mesh silica gel and 2% methanol-chloroform, v/v, as eluent) yielded H8 as a colourless gummy solid (96 mg, 22% yield, Rf=0.5 in 10% methanol-chloroform, v/v).

ESI-MS: m/z=743 [M⁺+1] for C₄₃H₆₆O₄N₃ClF.

ESI-HRMS: m/z=742.47204 for C₄₃H₆₆O₄N₃ClF.

¹H NMR (400 MHz, CDCl₃): δ/ppm=0.80-0.91 [t, 6H], 1.17-1.37 [m, 32H], 1.61-1.73 [m, 4H], 1.77-2.20 [m, 9H], 2.49-2.53 [m, 1H], 2.61-2.74 [m, 3H], 2.98-3.07 [m, 3H], 3.40 [s, 2H], 3.50 [m, 1H], 3.93 [s, 1H], 7.10-7.14 [t, 1H], 7.28-7.39 [m, 3H], 7.98-8.02 [m, 1H].

¹³C NMR (100 MHz, CDCl₃): δ/ppm=198.01, 169.71, 164.76, 162.44, 141.99, 130.62, 128.64, 126.23, 115.7, 115.5, 79.71, 62.87, 60.72, 56.99, 48.76, 49.09, 35.84, 34.98, 34.31, 33.73, 31.55, 29.68, 28.94, 26.11, 22.53, 22.24, 20.67, 14.10, 14.02.

### Example 7

### Synthesis of Q8 (g)

Compound (c) (0.100 g, 0.234 mmol) was dissolved in 5 mL of dry DMF in a 25 mL round-bottomed flask and stirred over an ice bath for 15 minutes. To this, N-[(Dimethylamino)-1H-1,2,3-triazolo-[4,5-b]pyridin-1-ylmethylene]-N-methylmethanaminium hexafluorophosphate N-oxide (HATU) (0.09 g, 0.25 mmol) was added, and stirring was continued for another 30 minutes. Then compound methyl-3-amino-propanoate (62 mg, 0.60 mmol) dissolved in 2 mL of dry DMF was added followed by dropwise addition of diisopropylethylamine (DIPEA) until the reaction mixture became slightly basic. The resulting mixture was stirred for 48 hours. Then the reaction mixture was dissolved in 20 mL of DCM, washed with 1N-HCl (2×20 mL), water (1×20 mL), and brine (1×20 mL), dried with anhydrous Na₂SO₄, and evaporated. Column chromatographic purification (using 60-120 mesh silica gel and 3% methanol-chloroform, v/v, as eluent yielding compound as a yellowish-brown gummy liquid (89.6% yield, Rf=0.6 in 10% methanol-chloroform, v/v).

ESI-MS: m/z=399.36 [M⁺] for C₂₂H₄₅O₃N₂Cl.

¹H NMR (500 MHz, CDCl₃): δ/ppm=0.86-0.92 [t, 6H], 1.21-1.40 [m, 21H], 1.60-1.72 [m, 4H], 2.05 [s, 1H], 3.33-3.37 [m, 3H], 3.45-3.68 [m, 7H], 4.18-4.23 [s, 2H].

### Example 8

Preparation of CSP conjugates: 3 mM of H8 and Q8 stocks in 5 mL of methanol (HPLC grade) were prepared separately. Those stock solutions were added to CSP (10 mg) individually and kept under bath sonication for 5 minutes followed by stirring for 12 hours at room temperature (RT). The nano conjugate mixture was centrifuged for 10 minutes at 10,000 rpm at 27□, the resulting CSP nano conjugate pellet was used for further characterization studies.

### Characterization of H8, Q8 and its CSP Conjugates

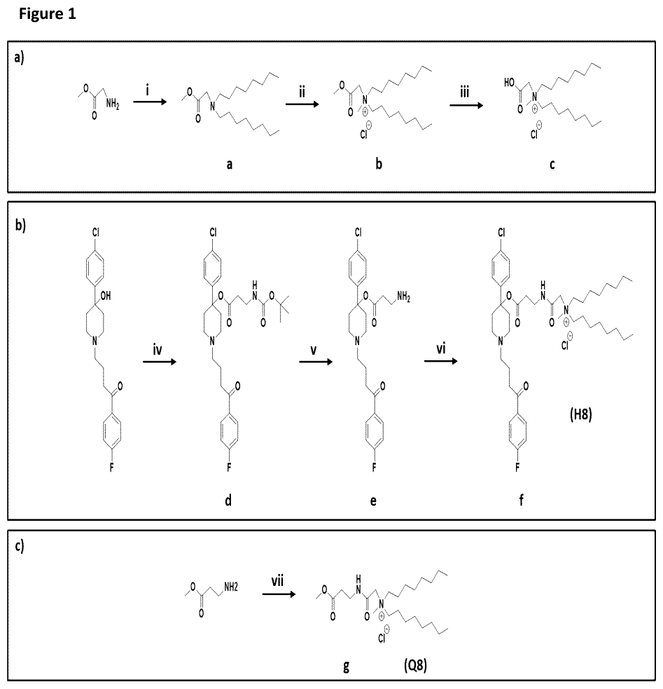

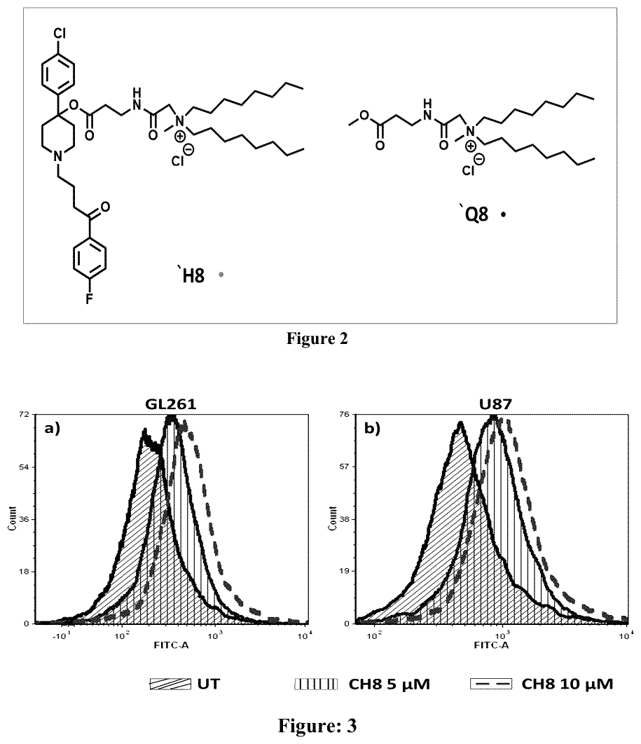

Chemical structures of H8 and Q8 cationic molecules (FIG. 1). CSP synthesis and association of chemical compounds on CSP was accomplished as per the protocol described earlier (Selvi et al., 2008). CSP was then associated with either H8, Q8 as per protocol discussed earlier (Indian Patent Application No. 201841009113; filed on Mar. 13, 2018) and were labelled as CH8 (CSP-H8) and CQ8 (CSP-Q8) respectively. These CSP conjugates were dispersed in aqueous solution and their hydrodynamic diameter (ranging from 340 to 380 nm), polydispersity index (PDI<0.3) and surface charges (−37 to −21 mV) were measured using zeta sizer (Table 1). The increases in zeta potential measurement from −37 to −23 and −22 mV respectively clearly indicate the adsorption of cationic lipids, H8 and Q8 respectively on CSP. Comparison of FT-IR spectrums of naive CSP and CSP conjugates with that of pure lipids indicates the attachment/association of lipids on CSP. The hydrodynamic diameter and PDI values state that CSP conjugates are homogenously distributed and is further supported by uniform distribution of spherical nano conjugates in FE-SEM images. Amount of H8 and Q8 adsorbed to CSP was measured by using HPLC at 27□. The data showed that about 1.8 mg (2.3 μmol) of H8 and 0.65 mg (1.9 μmol) of Q8 were adsorbed on 10 mg of CSP.

DLS Studies: The hydrodynamic diameter and surface charge of CSP, CH8 (CSP-H8), CQ8 (CSP-Q8), were measured using the instrument Anton-Paar litesizer-500. Briefly, 20 μL of CSP nano conjugate suspension was further diluted to 1 mL of deionised water. Then hydrodynamic diameter and surface charge was measured (Table 1).

### Example 9

### H8 Imparts SR-Targeted Cellular Uptake of CSP in GL261 and U87 Cells

To check the uptake study in GL261 (mouse glioma), U87 cells (human glioblastoma) and non-cancerous cells CHO and HEK293, the nanospheres CH8 and CQ8 were further conjugated with Rh-PE and were treated to these cells for 4 hours. FACS uptake analysis reveals more fluorescence shift towards right in CH8 treated brain cancer cells than the shifts obtained from cells treated by CQ8. Hence, presence of H8-ligand facilitated CH8 to get better admittance in both the cells GL261 and U87. It is clear that cytometry data depict more uptake of CH8-Rh-PE to brain tumor cells. Among these cells, U87 exhibits higher uptake of CH8 than in GL261 cells. This is in consonance with the fact that SR-expression is nearly similar in both the GL261 and U87, with U87 exhibiting little higher SR-expression than GL261. However, to know whether the uptake in brain cancer cells is SR-assisted or not, GL261 and U87 cells were pre-treated with SR-antagonist, haloperidol before CH8 treatment.

### Example 10

CH8 shows efficient and selective cancer cell killing: The cytotoxicity of H8, CH8 and CQ8 respectively were examined and compared in GL261, U87, HEK293 and CHO cells by 3-(4,5-dimethylthiazol-2-yl)-2,5-diphenyltetrazolium bromide (MTT) assay. CH8 shows IC50 of CH8 shows 1.9-2.3 μM whereas pristine H8 molecule shows IC50 at 2.0-2.6 μM and CQ8 showed insignificant killing at those concentration ranges in GL261 and U87 cells, indicating CH8 is significantly effective than CQ8 in killing cancer cells. Moreover, the cytotoxic effects of CH8, H8, CQ8 are negligible in those concentration ranges in non-cancer cells such as CHO, HEK293, where SR is basally or negligibly expressed.

### ROS Generation and Apoptosis

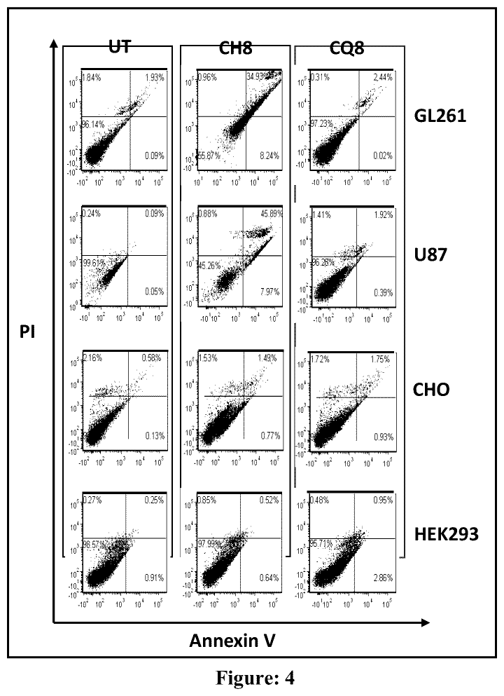

CH8 on treatment, produces ROS in GL261 and U87 cells and the production of ROS increased with increased concentration of CH8 (FIG. 3). Additionally, GL261 and U87 cells, upon treatment with CH8 exhibited significantly a greater number of late apoptotic cells on comparing the cells treated with CQ8. However, CH8 treatment led to insignificant population of late apoptotic cells in non-cancerous CHO and HEK293 cells. The data (FIG. 4) in overall indicates the selective apoptosis inducing ability of CH8 in cancer cells.

### Example 11

TABLE 1
Hydrodynamic size, Zeta potential and PDI of
CSP and its conjugate: CSP represents carbon
nanospheres; CH8 (CSP-H8) and CQ8 (CSP-Q8).

| | | Hydrodynamic | Zeta | |
|---|---|---|---|---|
| S No | Sample | diameter (nm) | potential(mV) | PDI |
| 1 | CSP | 344.94 ± 9.6 | −37 ± 1.1 | 0.25 ± 0.033 |
| 2 | CH8 | 357.53 ± 10.79 | −21.8 ± 0.3 | 0.23 ± 0.05 |
| 3 | CQ8 | 379.9 ± 7.2 | −23.4 ± 0.3 | 0.26 ± 0.01 |

### CH8 Accumulates in Orthotopic Glioma Tumor in Mice with Higher Efficiency Than CSP

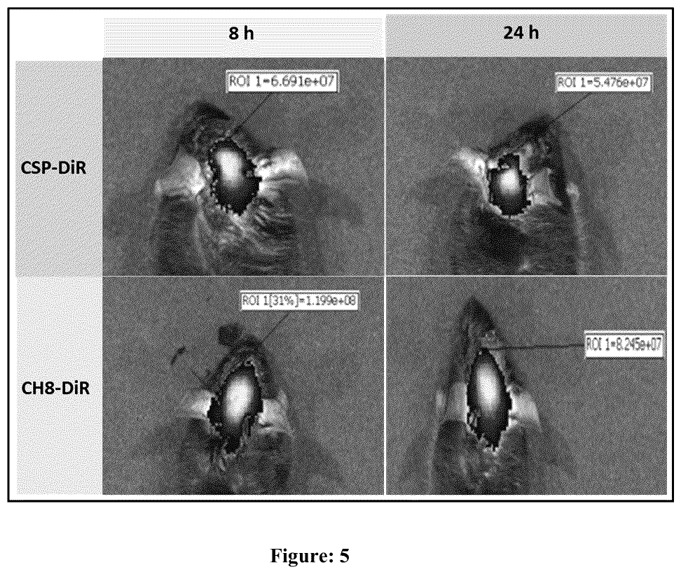

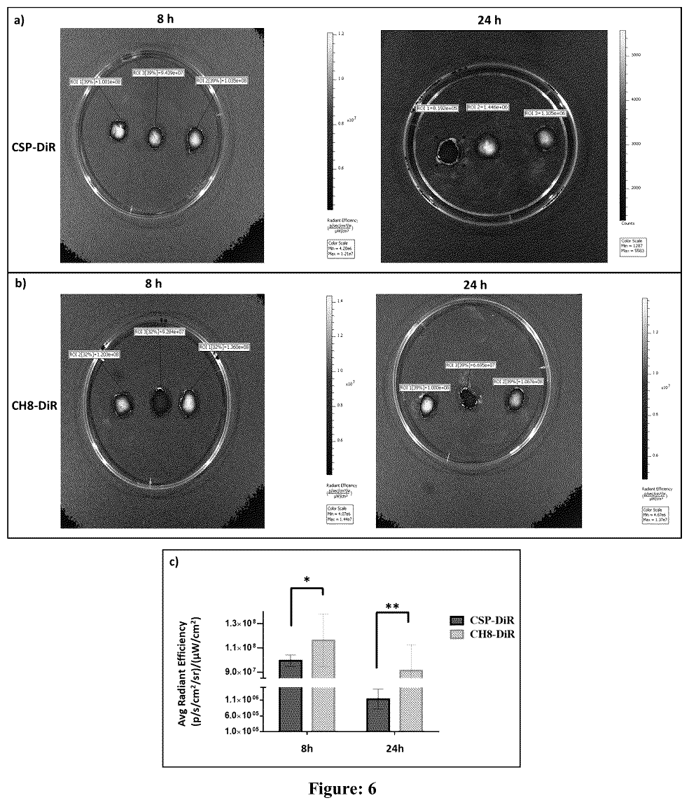

After confirming the selective cytotoxicity of CH8 towards cancer cells in in vitro studies, the accumulation of CH8 were examined in orthotopic glioma mouse model. For this, the CSP and CH8 surfaces are conjugated with NIR dye (DiR) and represented as CSP (CSP-DiR) and CH8 (CH8-DiR). Tissue distribution of (CSP-DiR) and (CH8-DiR) were examined following intraperitoneal injection in orthotopic GL261 tumor bearing C57BL/6J mice for two different time points (8 hours and 24 hours). The data from mice in vivo images on dorsal view show a greater number of nanomaterials accumulation in brain (FIG. 5). Significantly higher amount of CH8-DiR was accumulated in brain than CSP-DiR at both the time points. Although the NIR intensity in brain decreased for both the treatment groups after 24 hours, but the retention of CH8-DiR in brain was significantly higher than the accumulated amount of CSP-DiR in brain. This was further supported by ex vivo glioma brain images (FIG. 6). Clearly, CH8 exhibited enhanced permeability and retention compared to non-targeted CSP in brain indicating its potentially higher tendency to interact in situ glioma, if any. As the difference of accumulation is evident due to the presence and absence of H8, a SR ligand, it can be concluded that SR expression in brain can be exploited for effective targeting of nanoparticles to brain.

### Example 12

### Tumor Growth Inhibition and Survivability Studies

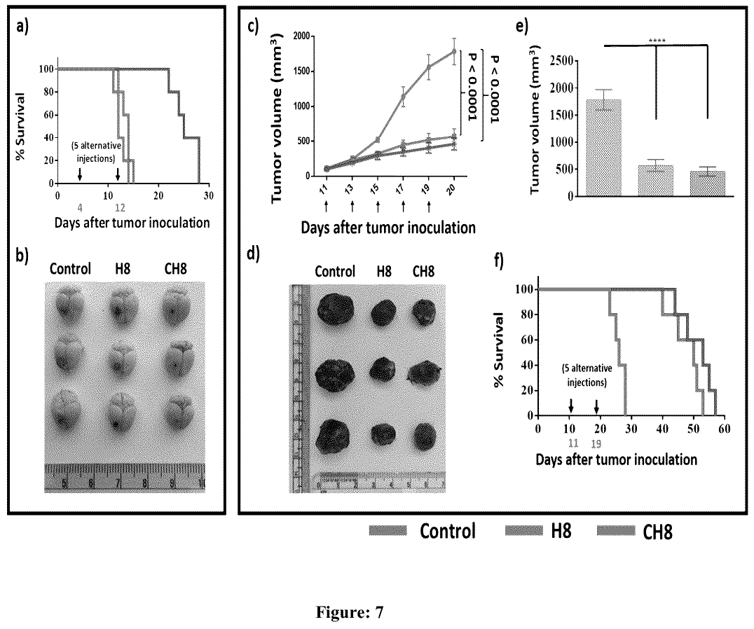

Further, to observe the orthotopic brain tumor growth inhibitory efficiency of presently described SR-targeting CH8, we checked survivability among brain tumor-bearing mice following 5 alternate dosing of various treatment groups. Herein, the intraperitoneal injections started on 4^{th} day until 12^{th} day (total 5 injections) since stereotactic-based inoculation of GL261 cells in brain. The CH8 treated mice shown a significant survivability on comparing with the mice treated with free H8 and untreated groups (FIG. 7a). In another set of experiment, animals bearing brain in situ GL261 cells and having 5 injections of various treatments were sacrificed on 12^{th} day to visualize the status of tumor lesions in full brains obtained from respective treatment groups. Brains from both UT and H8-treated mice showed comparable effect on tumor sites (black spots), which are visually much bigger than those in brains obtained from CH8-treated mice (FIG. 7b). Clearly, the visual effect of respective treatments has reflected on overall survivability of treated mice.

As evidenced earlier, H8 has its own anticancer effect, but possibly is unable to traverse through BBB to show its antitumor effect in mice with in-situ glioma tumor. If this notion is correct, in GL261 subcutaneous tumor model, the antitumor effect of H8 and CH8 should remain same. To prove this hypothesis, we developed the subcutaneous model and followed the same 5-injection treatment pattern which began on 11^{th} day post inoculation of GL261 cells. The similar antitumor effects of H8 and CH8 are evident from tumor regression curve (FIG. 7c). Moreover, visual images (FIG. 7d) and respective volumes (FIG. 7e) of tumors from different treatment groups obtained after sacrificing the mice on 20^{th} day post inoculation, substantiate the similar antitumor effects posed by H8 and CH8. These effects cumulatively affected the overall survivability of mice under different treatment groups, wherein we witnessed similar survivability of mice among groups treated respectively with H8 and CH8 (FIG. 7f).

### Example 13

### Isolation of TAMs and Estimation of SR Expression in TAMs Population Obtained From Tumor Microenvironment

Realizing higher survivability and visualizing relatively very small area of tumor lesions in isolated brains obtained from CH8-treated orthotopic tumor bearing mice, we presumed that the anticancer effect should be cumulatively on tumor microenvironment (TME)-associated tumor epithelial cells as well as on other pro-tumorigenic cells that release favourable factors in TME. TME contains many types of pro-tumorigenic factors secreted by various cells including tumor- associated macrophages (TAM). Hence, we chose to decipher if TAM could have been also targeted and killed by CH8. This is possible only when CH8 could be taken up efficiently, possibly through its cognate receptor SR, if expressed on TAM.

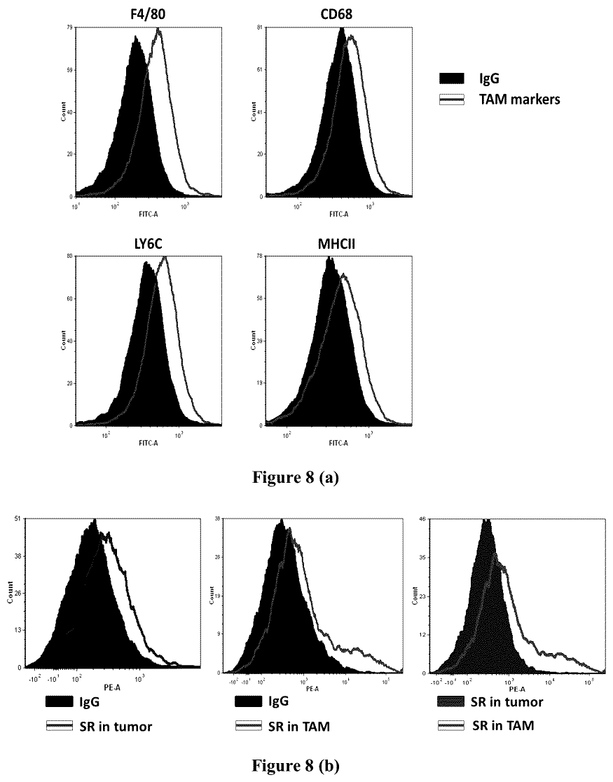

For that, as a proof of concept, TAMs were isolated by using MicroBeads against CD11b of tumor lysate of subcutaneous GL261 tumor. CD11b is one of the surface markers of TAM. Magnetically separated CD11b+ TAMs fraction was first checked to see if these are predominantly pro-tumorigenic M2 subtypes, using antibodies against various surface markers such as F4/80, CD68 (FIG. 8a), which are well known marker for M2-macrophage. Clearly, there were more expression of F4/80 and CD68 in TAMs. In particular, lack of LY6C and low MHCII surface marker expressions were also recorded, which characteristically represent that the TAM so isolated are either devoid of or are having less levels of marker for anti-tumorigenic macrophages M1. Clearly, the population of TAMs so obtained contains activated macrophages (M2 or TAMs), which can suppress antitumor immunity and promote tumor growth.

After isolation of TAM and also segregating the TAM-free tumor cell fraction, we checked for the expression of SR both in TAMs and isolated tumor cells by FACS analysis. We find that SR is expressed in both cell types. But surprisingly SR is expressed more in TAMs compared to isolated tumor cells (FIG. 8b).

### Example 14

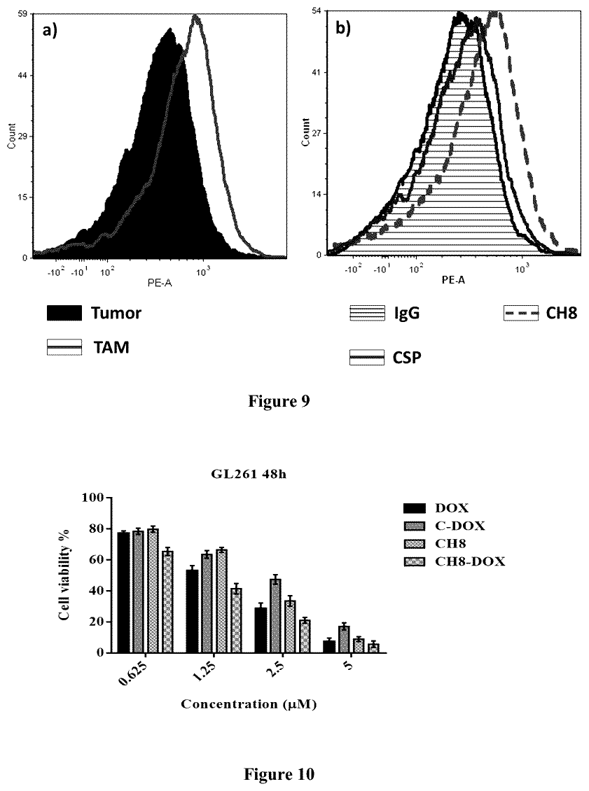

CH8 accumulates in TAM in-vivo: For this, after GL261 subcutaneous tumor inoculation, when the tumor volume is 1500 mm³, mice were separated into two sets and individually treated with Rh-PE conjugated CH8 and CSP. After 8 h, TAMs and tumor cells were collected from subcutaneous tumors of sacrificed mice under different treatment groups. FACS study reveals higher uptake of Rh-PE in TAMs from CH8 treated mice compared than in isolated tumor cells (FIG. 9a). FIG. 9b, clearly shows that TAMs took up more CH8 than CSP, indicating that as TAMs express SR, SR-targeted nanosphere has increased ability to reside in TAMs in a given time point.

Cytotoxicity of CH8-DOX in GL261 cells: The cytotoxicity of DOX, CSP-DOX (C-DOX), CH8 and CH8-DOX respectively were examined and compared in GL261 cells by 3-(4,5-dimethylthiazol-2-yl)-2,5-diphenyltetrazolium bromide (MTT) assay. For the given concentration of DOX, the cytotoxic effect of CH8-DOX is comparatively higher than the DOX, C-DOX and also in the presence of corresponding concentration of CH8 nano-conjugates (FIG. 10).

### ADVANTAGES OF THE PRESENT INVENTION

Developing a potent targeting carrier with enhanced therapeutic efficacy towards deadly cancer like glioblastoma. The crossing of BBB to reach the brain cancer cells was playing a key role in making an efficient therapeutic drug. Herein receptor targeted ligand which acts as anti-proliferative against brain cancer cells in low concentrations, helps in selective killing of tumor cells. Furthermore, accommodating an approved anti-cancer drug showing an prominent cytotoxic effect in very low concentration.

1.-18. (canceled)

19. A nanoformulation having anticancer activity, the nanoformulation comprising:
a complex, the complex comprising:
a carbon nanosphere (CSP); and
a sigma receptor targeting ligand (H8);
wherein the carbon nanosphere and the sigma receptor targeting ligand are in a ratio of 1:0.08 to 1:0.2.

20. The nanoformulation as claimed in claim 19, wherein the carbon nanosphere (CSP) is glucose.

21. The nanoformulation as claimed in claim 19, wherein the sigma receptor targeting ligand is haloperidol derivative and is H8; wherein the H8 is haloperidol chemically conjugated with cationic lipids of twin-chain aliphatic carbon chains of C8 length.

22. The nanoformulation as claimed in claim 19, wherein the nanoformulation (CSP-H8) is further conjugated with a hydrophilic or hydrophobic drug (D), wherein the drug is selected from the group of anticancer drugs consisting of doxorubicin, gemcitabine, temozolomide, carmustine, and everolimus.

23. The nanoformulation as claimed in claim 19, wherein the nanoformulation is characterized for targeting tumor epithelial cell (TEC) and tumor associated macrophages (TAM) in glioblastoma mass.

24. A process for the preparation of a nanoformulation having anticancer activity, the process comprising:
i.) dissolving N-(carboxymethyl)-N-methyl-N-octyloctan-1-aminium chloride in dry dimethylformamide (DMF), and stirring over an ice bath to obtain a mixture;
ii.) adding N-[(Dimethylamino)-1H-1,2,3-triazolo-[4,5-b] pyridine-1-ylmethylene]-N-methylmethanaminium hexaflurophosphate N-oxide (HATU) to the mixture to obtain a reaction mixture;
iii.) dissolving β-Alanine-Haloperidol conjugate in dry dimethylformamide (DMF) to obtain a conjugate mix;
iv.) adding diisopropylethylamine (DIPEA) and the conjugate mix dropwise to the reaction mixture obtained in step (iii), until the reaction mixture becomes basic;
v.) stirring the reaction mixture as obtained in step iv for 40-50 hours;
vi.) dissolving dichloromethane (DCM) in the reaction mixture followed by washing with 1N-HCl, water and brine, drying with anhydrous Na2SO4, evaporating, and purifying to obtain H8;
vii.) dissolving H8 in methanol to obtain a solution, and adding the solution to CSP and keeping the conjugate mixture of CSP-H8 under bath sonication for 5-10 minutes followed by stirring for 10-12 hours at room temperature to obtain a nanoconjugate mixture; and
viii.) centrifuging the nanoconjugate mixture for 10 minutes at 20-30° C., to obtain the nanoformulation in form of CSP-H8 nanoconjugate pellet.

25. The process as claimed in claim 24, wherein the nanoformulation (CSP-H8 nanoconjugate pellet) is further conjugated with a hydrophilic or hydrophobic drug (D), wherein the drug is selected from the group of anticancer drugs consisting of doxorubicin, gemcitabine, temozolomide, carmustine, and everolimus.

26. The process as claimed in claim 24, wherein the nanoformulation is characterized for targeting tumor epithelial cell (TEC) and tumor associated macrophages (TAM) in glioblastoma mass.

27. A process comprising:
(i) conjugating a carbon nanosphere (CSP) with a sigma receptor targeting ligand (H8) to form a CSP-H8 or CH8 nanoconjugate; and
(ii) conjugating a potent drug D to the CSP-H8 or CH8 nanoconjugate by mixing CSP-H8 or CH8 nanoconjugate with an alcoholic solution of the potent drug D and stirring for a period sufficient to ensure linking of D to CH8 with a maximum limit maintaining a CH8:D ratio of 1:0.2.

28. The process as claimed in claim 27, wherein the period of stirring is 7-15 hours.

29. The process as claimed in claim 27, wherein the alcohol used is C1 to C3 alcohol.

30. A drug delivery kit for specific delivery of a drug molecule to a tumor site, having a nanoformulation as claimed in claim 19.

31. The drug delivery kit as claimed in claim 30, wherein the nanoformulation is further conjugated with a hydrophilic or hydrophobic drug (D), wherein the drug is selected from a group of anticancer drugs consisting of doxorubicin, gemcitabine, temozolomide, carmustine and everolimus.

32. The drug delivery kit as claimed in claim 30, wherein the kit is configured for targeting tumor epithelial cell and tumor associated macrophages for treatment of glioblastoma or tumor mass.

33. A method of treating a tumor or glioblastoma mass by targeting tumor epithelial cells (TEC) and tumor-associated macrophages (TAM) with the nanoformulation as claimed in claim 19.

34. A method of treating tumor or glioblastoma mass by targeting tumor epithelial cells (TEC) and tumor-associated macrophages (TAM) with the drug delivery kit as claimed in claim 30.
